(19) United States
(12) Patent Application Publication
(10) Pub. No.: US 2008/0262057 A1
(43) Pub. Date: Oct. 23, 2008

(54) Diarylisoxazolines

(75) Inventors: Francis E. Tisdell, Carmel, IN (US); William C. Lo, Fishers, IN (US); Joseph D. Eckelbarger, Carmel, IN (US); Ricky Hunter, Westfield, IN (US); Noormohamed M. Niyaz, Indianapolis, IN (US); James M. Renga, Indianapolis, IN (US); Gregory T. Whiteker, Carmel, IN (US)

Correspondence Address: DOW AGROSCIENCES LLC, 9330 ZIONSVILLE RD, INDIANAPOLIS, IN, 46268, US

(73) Assignee: Dow AgroSciences LLC, Indianapolis, IN (US)

(21) Appl. No.: 12/148,540

(22) Filed: Apr. 18, 2008

Related U.S. Application Data
(60) Provisional application No. 60/925,369, filed on Apr. 20, 2007.

(51) Int. Cl. A01N 43/80; C07D 261/04; A01C 1/06

(52) U.S. Cl. 514/378; 548/240

(57) ABSTRACT

The invention disclosed in this document is related to field of pesticides and their use in controlling pests.

### CROSS-REFERENCE TO RELATED APPLICATIONS

This application claims priority from U.S. Provisional Application Ser. No. 60/925,369 filed on Apr. 20, 2007, the disclosure of which is hereby incorporated by reference.

### FIELD OF THE INVENTION

The invention disclosed in this document is related to field of pesticides and their use in controlling pests.

### BACKGROUND OF THE INVENTION

Pests cause millions of human deaths around the world each year. Furthermore, there are more than ten thousand species of pests that cause losses in agriculture. These agricultural losses amount to billions of U.S. dollars each year. Termites cause damage to various structures such as homes. These termite damage losses amount to billions of U.S. dollars each year. As a final note, many stored food pests eat and adulterate stored food. These stored food losses amount to billions of U.S. dollars each year, but more importantly, deprive people of needed food.

There is an acute need for new pesticides. Insects are developing resistance to pesticides in current use. Hundreds of insect species are resistant to one or more pesticides. The development of resistance to some of the older pesticides, such as DDT, the carbonates, and the organophosphates, is well known. But resistance has even developed to some of the newer pesticides. Therefore, a need exists for new pesticides and particularly for pesticides that have new modes of action.

### Substituents

### Non-Exhaustive List

The examples given for the substituents are (except for halo) non-exhaustive and must not be construed as limiting the invention disclosed in this document.

“alkenyl” means an acyclic, unsaturated (at least one carbon-carbon double bond), branched or unbranched, substituent consisting of carbon and hydrogen, for example, vinyl, allyl, butenyl, pentenyl, hexenyl, heptenyl, octenyl, nonenyl, and decenyl.

“alkoxy” means an alkyl further consisting of a carbon-oxygen single bond, for example, methoxy, ethoxy, propoxy, isopropoxy, 1-butoxy, 2-butoxy, isobutoxy, tert-butoxy, pentoxy, 2-methylbutoxy, 1,1-dimethylpropoxy, hexoxy, heptoxy, octoxy, nonoxy, and decoxy.

“alkyl” means an acyclic, saturated, branched or unbranched, substituent consisting of carbon and hydrogen, for example, methyl, ethyl, propyl, isopropyl, 1-butyl, 2-butyl, isobutyl, tert-butyl, pentyl, 2-methylbutyl, 1,1-dimethylpropyl, hexyl, heptyl, octyl, nonyl, and decyl.

“aryl” means a cyclic, aromatic substituent consisting of hydrogen and carbon, for example, phenyl, naphthyl, and biphenylyl.

“cycloalkyl” means a monocyclic or polycyclic, saturated substituent consisting of carbon and hydrogen, for example, cyclopropyl, cyclobutyl, cyclopentyl, cyclohexyl, cycloheptyl, cyclooctyl, cyclodecyl, norbornyl, bicyclo[2.2.2]octyl, and decahydronaphthyl.

“halo” means fluoro, chloro, bromo, and iodo.

“haloalkyl” means an alkyl further consisting of, from one to the maximum possible number of, identical or different, halos, for example, fluoromethyl, difluoromethyl, trifluoromethyl, 1-fluoroethyl, 2-fluoroethyl, 2,2,2-trifluoroethyl, chloromethyl, trichloromethyl, and 1,1,2,2-tetrafluoroethyl.

“heterocyclyl” means a cyclic substituent that may be fully saturated, partially unsaturated, or fully unsaturated, where the cyclic structure contains at least one carbon and at least one heteroatom, where said hertoatom is nitrogen, sulfur, or oxygen, for example, thiophene, furan, pyrrole, thiazole, oxazole, imidazole, isothiazole, isoxazole, pyrazole, 1,3,4-oxadiazole, 1,3,4-thiadiazole, 1,3,4-triazole, 1,2,4-oxadiazole, 1,2,4-thiadiazole, 1,2,4-triazole, 1,2,3-triazole, 1,2,3,4-tetrazole, benzo[b]thiophene, benzo[b]furan, indole, benzo[c]thiophene, benzo[c]furan, isoindole, benzoxazole, benzothiazole, benzimidazole, benzisoxazole, benzisothiazole, benzopyrazole, benzothiadiazole, benzotriazole, dibenzofuran, dibenzothiophene, carbazole, pyridine, pyrazine, pyrimidine, pyridazine, 1,3,5-triazine, 1,2,4-triazine, 1,2,4,5-tetrazine, quinoline, isoquinoline, quinoxaline, quinazoline, cinnoline, 1,8-naphthyridine, 1,5-naphthyridine, 1,6-naphthyridine, 1,7-naphthyridine, phthalazine, pyridopyrimidine, purine, pteridine, 4H-quinolizine, piperidine, morpholine, piperazine, oxetane, oxirane, pyrrolidine, oxazoline, tetrahydrofuran, tetrahydropyran, isoxazolidine, and thiazolidine.

### DETAILED DESCRIPTION OF THE INVENTION

The inventive compounds have the general formula

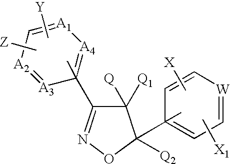

wherein:

A₁, A₂, A₃, and A₄ are independently C or N;

Q, Q₁, and Q₂, are independently, H, (C₁-C₆)alkyl, halo(C₁-C₆)alkyl, halo, CN, CO₂R, and CONR₁R₂;

W is C or N;

X and X1 are independently, H, halo, CN, NO₂, (C₁-C₆)alkyl, (C₁-C₆)alkyl-S—, (C₁-C₆)alkoxy, halo(C₁-C₆)alkoxy, halo(C₁-C₆)alkyl-S—, (C₂-C₆)alkenyl, and SCN;

Y and Z are independently, H, halo, CN, NO₂, (C₁-C₆)alkyl, (C₁-C₆)alkyl-S—, (C₁-C₆)alkoxy, halo(C₁-C₆)alkoxy, halo(C₁-C₆)alkyl-S—, (C₂-C₆)alkenyl, SCN, NRR₁, OR₁, or D₁, D₂, D₃, D₄, D₅, D₆, D₇, D₈, D₉, D₁₀, and D₁₁

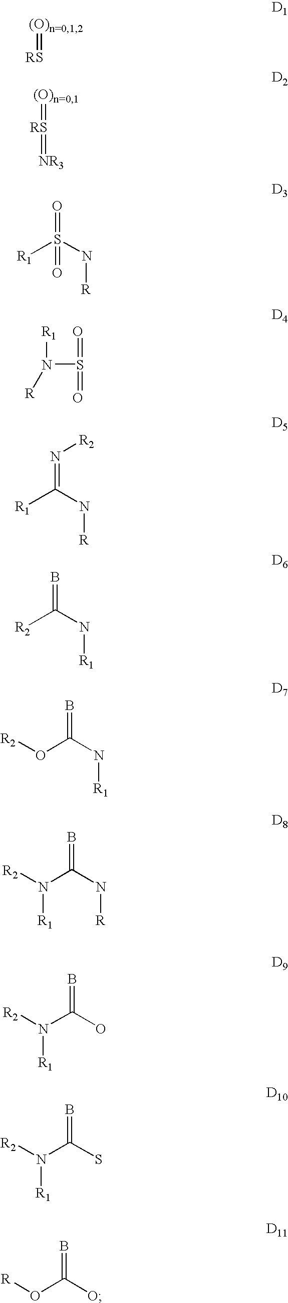

B is O or S;

R, R₁, and R₂, are independently, H, (C₁-C₆)alkyl, halo(C₁-C₆)alkyl, (C₃-C₆)cycloalkyl, (C₂-C₆)alkenyl, halo(C₂-C₆)alkenyl, aryl, heterocyclyl, aryl-alkyl-, heterocyclyl-alkyl-;

R₁ and R₂ can be connected by C, N, O, or S, to form a 3-6 membered ring; and

R₃ can be H, NO₂, CN, COH, COR₁, and CO₂R₁.

In another embodiment of this invention Y is D₁ or D₂.

In another embodiment of this invention Q and Q₁ are H.

In another embodiment of this invention Q₂ is a halo(C₁-C₆)alkyl.

In another embodiment of this invention W is C.

In another embodiment of this invention X and X₁ are halo.

In another embodiment of this invention A₁, A₂, A₃, and A₄ are C.

In another embodiment of this invention R is a (C₁-C₆)alkyl.

In another embodiment of this invention Z and Y are H.

In another embodiment of this invention R₃ is H, CN, NO₂, COR₁, or COOR₁.

### General Synthesis Scheme

The inventive compounds disclosed in this document can be made as follows.

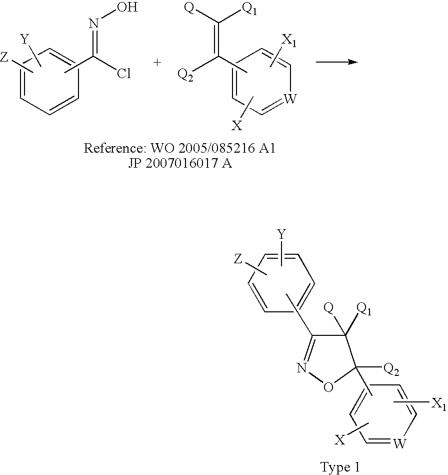

### General Procedures for the Preparation of Isoxazolines of Type 1

### Method A

To a 25 mL round bottom flask containing benzohydroximinoyl chloride (prepared according to Journal of Organic Chemistry, 1980, 45, 3916-3918; JOC, 1992, 57(24), 6649-6650) (1.2 mmol), α-(trifluoromethyl)styrene (prepared according to Journal of Fluorine Chemistry 95 (1999) 167-170; EP 1 731 512 A1) (1.0 mmol) and 10 mL of 10% aqueous tetrahydrofuran was added solid KHCO₃ (2.4 mmol) in one portion. The resulting reaction mixture was stirred at room temperature for 18 h. The reaction mixture was added to H₂O and extracted with EtOAc, dried (MgSO₄), and concentrated to give the crude isoxazoline product as either an oil or solid. The crude solid was washed with 20% Et₂O/Hexane to give sufficient purity (>95%). When the crude product was an oil, it was purified via flash chromatograph.

### Method B

To a 25 mL round bottom flask containing benzaldoxime (2.41 mmol) in 15 mL of dimethylformamide was added N-Chlorosuccinimde (2.41 mmol) in one portion. The resulting reaction mixture was stirred at room temperature for several hours to give benzohydroximinoyl chloride. After which time, α-(trifluoromethyl)styrene (2.48 mmol) and KHCO₃ (2.65 mmol) were added to the reaction mixture. Reaction mixture continued to stir at room temperature for 18 h. The reaction mixture was added to H₂O and extracted with EtOAc, dried (MgSO₄), and concentrated to give the crude isoxazoline product as either an oil or solid. The crude product was washed with 20% Et₂O/Hexane to give sufficient purity (>95%). When the crude product was an oil, it was purified via flash chromatograph.

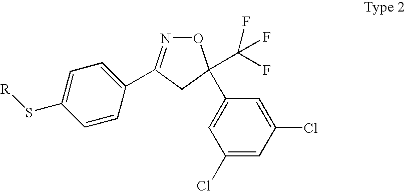

### General Procedure for the Preparation of Isoxazolines of Type 2

### Method C

Following a literature procedure (Tetrahedron 62 (2006) 2357-2360), a 250 mL round bottom flask containing R—SH (15.6 mmol; R as defined above) and dimethylsulfoxide (90 mL) was added Cs₂CO₃ (14.85 mmol). The resulting reaction mixture was stirred at room temperature for 0.5 h. After which time, 5-(3,5-dichlorophenyl)-3-(4-NO₂-phenyl)-5-trifluoromethyl-4,5-dihydroisoxazole (7.43 mmol, prepared by Method A or B) was added as solid in small portions to give a dark green solution. After 0.5 h, reaction was complete as indicated by thin layer chromatography (tlc). The reaction mixture was added to H₂O and extracted with Et₂O. Organic layer was washed several times with H₂O, dried (MgSO₄), and concentrated to give the crude isoxazoline product as either an oil or solid. The crude product was purified via flash chromatograph eluting first with hexane (2 L), then 3% Et₂O/Hexane to give the desired product as either an oil or a solid.

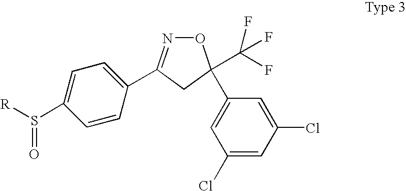

### General Procedure for the Preparation of Isoxazolines of Type 3

Following a literature procedure (Tetrahedron Lett. 1983, 24, 1505-1508), a 50 mL round bottom flask containing a compound of Type 2 (4.77 mmol) and glacial acetic acid (20 mL) at 0° C. was added NaBO₃.4H₂O (4.77 mmol) in one portion. The resulting reaction mixture was allowed to warm toward room temperature and stirred for 18 h. The reaction mixture was added to Et₂OAc and washed carefully with aq. NaHCO₃, dried (MgSO₄), and concentrated to give a white solid. The crude product was washed with 20% Et2O/Hex to give sufficient purity (>95%). When the crude product was an oil, it was purified via flash chromatograph.

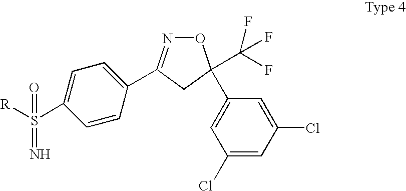

### General Procedure for the Preparation of Isoxazolines of Type 4

Following a literature procedure (Organic Letters, 2004, vol. 6, No. 8, 1305-1307), a 100 mL round bottom flask containing a compound of Type 3 (4.05 mmol), CF₃CONH₂ (8.1 mmol), PhI(OAc)₂ (6.07 mmol), MgO (16.18 mmol), and Rh₂(OAc)₄ (2.5 mole %) and 30 mL of CH₂Cl₂ was stirred at room temperature for 18 h. Reaction mixture was added to EtOAc and washed with H₂O. The aq. phase was back extracted with EtOAc. The combined organic layers were dried (MgSO₄), and concentrated to give an oil. The crude product was purified via flash chromatography to give the N-trifluoroacetyl intermediate as an oil. This material was dissolved in MeOH (15 mL) and K₂CO₃ (5 eq) was added in one portion. The reaction mixture was stirred at room temperature for 0.5 h. After which time, it was added to EtOAc and washed with H₂O. The aq. phase was back extracted with EtOAc. The combined organic layers were dried (MgSO₄), and concentrated to give a white solid. The crude product was washed with 20% Et₂O/Hex to give sufficient purity (>95%).

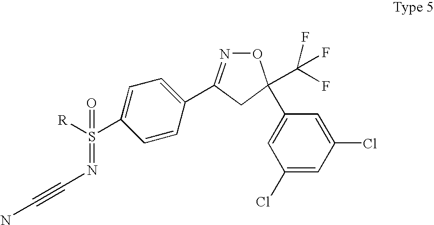

### General Procedure for the Preparation of Isoxazolines of Type 5

Following a procedure outlined in US200528027, a 25 mL round bottom flask containing a compound of Type 4 (0.44 mmol), 4-Dimethylaminopyridine (0.51 mmol) and 15 mL of CH₂Cl₂ was added BrCN (0.88 mmol) via syringe. The resulting reaction mixture was stirred at room temperature for 18 h. The reaction mixture was added to EtOAc, washed with 0.1N HCl, then aq. NaHCO₃, dried (MgSO₄), and concentrated to give the crude isoxazoline product as an oil. The crude material was purified via flash chromatograph eluting with 30% EtOAc/Hexane to give product as a thick oil.

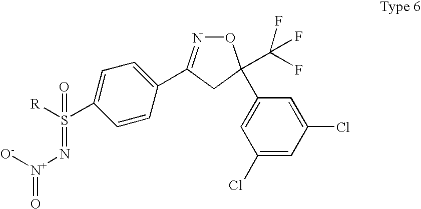

### Procedure for the preparation of Isoxazolines of Type 6 Following a procedure outlined in US200528027, a 25 mL round bottom flask containing a compound of Type 4 (0.33 mmol) and 3 mL of CH₂Cl₂ was added fuming 98% HNO₃ (0.33 mmol) then acetic anhydride (0.99 mmol) at 0° C. The resulting reaction mixture was allowed to warm toward room temperature and stirred for 2 h. The reaction mixture was added to EtOAc, washed with 0.5N NaOH, dried (MgSO₄), and concentrated to give product as a white foam with sufficient purity (>95%). The crude material, if necessary, could be purified via flash chromatograph.

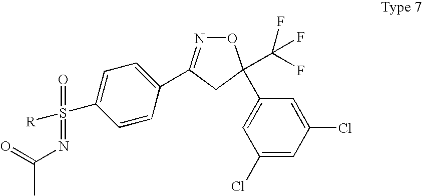

### Procedure for the preparation of Isoxazolines of Type 7

Following a literature procedure (Heterocycles, 1998, 49, 181), a 10 mL round bottom flask containing a compound of Type 4 (0.27 mmol) and 1-2 mL of pyridine was added acetic anhydride (1.35 mmol) at 0° C. The resulting reaction mixture was allowed to warm toward room temperature and stirred for 4 h. The reaction mixture was added to EtOAc, washed several times with 0.5N HCl then washed with aq. NaHCO₃, dried (MgSO₄), and concentrated to give an oil. It was triturated with Et₂O/Hex to give product as a white solid. The crude material, if necessary, could be purified via flash chromatography.

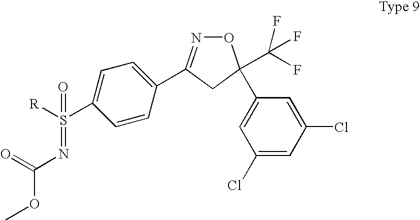

### Procedure for the preparation of Isoxazolines of Type 8

Following a literature procedure (Heterocycles, 1998, 49, 181), a 10 mL round bottom flask containing a compound of Type 4 (0.27 mmol) and 1-2 mL of pyridine was added methyl chloroformate (1.35 mmol) at 0 C. The resulting reaction mixture was allowed to warm toward room temperature and stirred for 4 h. The reaction mixture was added to EtOAc, washed several times with 0.5N HCl then washed with aq. NaHCO₃, dried (MgSO₄), and concentrated to give product as an oil (>95% pure). The crude material, if necessary, could be purified via flash chromatograph.

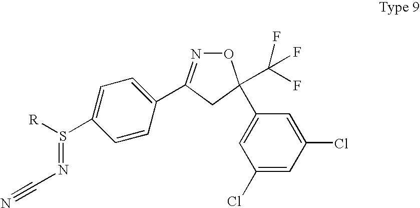

### Procedure for the preparation of Isoxazolines of Type 9

Following a literature procedure (Org. Lett. 2007, 9, 3809-3811), a 25 mL round bottom flask containing a compound of Type 2 (0.69 mmol) and 5 mL of CH₂Cl₂ was sequentially added cyanamide (0.90 mmol), solid potassium tert-butoxide (0.83 mmol) and NBS (1.0 mmol) to give a homogeneous bright yellow solution. The reaction mixture was stirred at room temperature for 0.5 h. Aq. sodium thiosulfate (˜5 μL) was added and the resulting reaction mixture was added to H₂O, extracted with CH₂Cl₂, dried (MgSO₄), and concentrated to give product as a white foam (>95% purity). The crude material, if necessary, could be purified via flash chromatograph.

### EXAMPLES

The examples are for illustration purposes and are not to be construed as limiting the invention disclosed in this document to only the embodiments disclosed in these examples.

### Compound Production

The following compounds were produced in accordance with the synthesis scheme.

### General Structure for Compounds in Table 1

TABLE 1

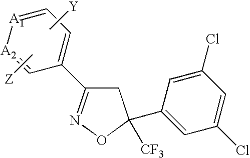

| Compound | Z | Y | A₁ | A₂ | mp ° C. | MS |
|---|---|---|---|---|---|---|
| 1 | 4-CF₃ | H | C | C | 101-2 | 427 |
| 2 | 4-I | H | C | C | | 483.9 |
| | | | | | | (M − 1) |
| 3 | 4-CF₃SO | H | C | C | 138-9 | 476 |
| | | | | | | (M + 1) |
| 4 | 4-CF₃SO₂ | H | C | C | | 491 |
| 5 | 4-CN | H | C | C | 137-8 | 384 |
| 6 | 4-SCF₃ | H | C | C | 108-9 | 459 |
| 7 | 4-Br | H | C | C | | 437.95 |
| | | | | | | (M − 1) |
| 8 | 4-OCF₃ | H | C | C | 69-70 | |
| 9 | 4-MeSO₂ | H | C | C | 170-1 | 437 |
| 10 | 4-MeSO₂N | H | C | C | 202 | 451 |
| | | | | | | (M − 1) |
| 11 | 4-iPrCON | H | C | C | 180-2 | 444 |
| 12 | 4-NH₂ | H | C | C | 109-111 | 374 |
| 13 | 4-NO₂ | H | C | C | 153-4 | 404 |
| 14 | 4-CF₃CH₂CON | H | C | C | 145-150 | 483 |
| | | | | | | (M − 1) |
| 15 | 4-F | H | C | C | | 376 |
| | | | | | | (M − 1) |
| 16 | 4-CF₃CH₂O | H | C | N | 130-1 | 459 |
| | | | | | | (M + 1) |
| 17 | 3-Br | H | C | C | | 437.94 |
| | | | | | | (M − 1) |
| 18 | 2-Cl | 4-Cl | C | C | | 427.98 |
| | | | | | | (M − 1) |
| 19 | 3-Cl | H | C | C | | 394.02 |
| | | | | | | (M − 1) |
| 20 | 4-iPr | H | C | C | | 402 |
| | | | | | | (M + 1) |
| 21 | 2-Cl | 5-Cl | N | C | 162-4 | |
| 22 | 4-CH₃ | H | C | C | | 373 |
| 23 | H | H | C | C | | 360.1 |
| | | | | | | (M + 1) |
| 24 | 2-NO₂ | H | C | C | 126-7 | 404 |
| 25 | 3-Cl | 5-Cl | C | C | 129.3 | |
| 26 | 3-NO₂ | H | C | C | 124-5 | 404 |
| 27 | 4-OCH₃ | H | C | C | 95-6 | 389 |
| 28 | 2-NH₂ | H | C | C | 90-2 | 374 |
| 29 | 3-OCH₃ | 4-F | C | C | | 406.05 |
| | | | | | | (M − 1) |
| 30 | 3-NH₂ | H | C | C | 93-5 | 374 |

### General Structure for Compounds in Table 2

TABLE 2

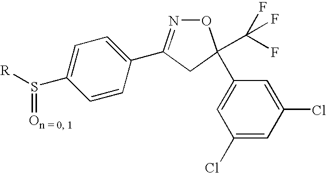

| Compound | R | n | mp ° C. | MS |
|---|---|---|---|---|
| 32 | Me | 0 | 113-6 | 405.8 (M + 1) |
| 32 | Et | 0 | | 419.74 (M + 1) |
| 33 | i-Pr | 0 | | 433 |
| 34 | Bn | 0 | 119-120 | 482 (M + 1) |
| 35 | Me | 1 | | |
| 36 | Et | 1 | | |
| 37 | i-Pr | 1 | 128-9 | 450.1 (M + 1) |
| 38 | Bn | 1 | 135-7 | |

TABLE 3

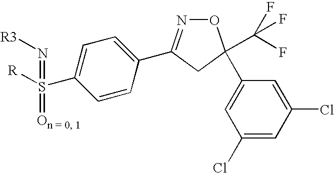

| Compound | R | R3 | n | mp ° C. | MS |
|---|---|---|---|---|---|
| 39 | Me | H | 1 | | 437 (M + 1) |
| 40 | Et | H | 1 | 148-9 | 448.78 (M − 1) |
| 41 | Bn | H | 1 | 66-7 | |
| 42 | Me | CN | 1 | | 460 (M − 1) |
| 43 | Et | CN | 1 | | 473.74 (M − 1) |
| 44 | i-Pr | CN | 1 | | 490.1 (M + 1) |
| 45 | Bn | CN | 1 | 80-2 | |
| 46 | Et | CN | 0 | | 460.0 (M + 1) |
| 47 | i-Pr | CN | 0 | | 474.0 (M + 1) |
| 48 | Et | NO2 | 1 | | 493.9 (M − 1) |
| 49 | Et | CH3CO | 1 | 200-2 | 490.73 (M − 1) |
| 50 | Et | MeOCO | 1 | | 507 (M − 1) |
| 51 | Me | CF3CO | 1 | | |
| 52 | Et | CF3CO | 1 | | 557.1 (M + 1) |
| 53 | Bn | CF3CO | 1 | | |

### Biological Testing

Insecticidal test for corn earworm (Helicoverpa zea) and beet armyworm (Spodoptera exigua). To prepare test solution, the test compound was formulated at 2000 ppm solution in 2 mL of 9:1 mixture of acetone:tap water. 50 μL of the test solution (50 μg/cm² dose for each well) was pippetted upon the surface of 1 mL of lepidopteran diet (Southland Multi-Species Lepidopteran Diet) contained in each of eight wells per insect species (one well=1 replication). A second-instar corn earworm and beet armyworm was placed upon the treated diet in each well once the solvent had air-dried. Trays containing the treated diet and larvae were covered and then held in a growth chamber at 25° C., 50-55% RH, and 16 hr light:8 hr dark for 5 days. Observations were conducted 5 days after treatment and infestation. The number of dead insects of 8 per species per treatment was then determined.

Insecticidal test for cotton aphid (Aphis gossypii) in foliar spray assay. Wingless cotton aphid, Aphis gossypii, nymphs and adults are cultured continuously in-house on squash plants (Pic-n-Pic variety). Newly non-infested squash plants with fully expanded cotyledon leaf stage are collected from the greenhouse and trimmed back to one cotyledon per plant (4 plants/replicates per treatment). Pots with non-infested squash plants are set-up in sets of 4 after being prepped and placed on aluminum trays on wheels. Plants are positioned in 3×9 rows (4 pots per set/treatment). The cotton aphid is infested 1 day prior to chemical application on newly collected plants by using cut infested leaves from the Insectary culture targeting 30-70 aphids per cut section. Aphid populated leaf sections are placed on top of cotyledon that will be sprayed. One day after infestation, aphids have migrated to the new plant that will be used for chemical application. Each plant is examined before chemical application to ensure ample infestation (ca. 30-70 aphids per plant). Leaf debris that was infested with aphids is removed from the newly infested plants before chemical is applied. Test solutions were prepared in a 25-ml glass vial. Each vial contained 2-mg technical grade test material. A stock solution was prepared by adding 2 ml of 50:50 solvent (acetone:methanol) to dissolve technical grade compound, forming a 1 mg/ml (1000 ppm). When dissolving the technical grade in solvent was problematic, the material was sonicated to facilitate the dissolving process before adding 0.025% Tween 20 in H₂O. Once technical material was dissolved, 8 ml of 0.025% Tween 20 in H₂O was added to achieve the 200 ppm high dose used in the test. Subsequent and final 50 ppm test dose used in the test was achieved by adding 2 ml of 200 ppm solution plus 6 ml of diluent (800 ml 0.025% Tween 20 in H₂O plus 200 ml acetone:methanol). Respective test solutions were applied to individual plants infested with the cotton aphid using a hand-held Devilbiss sprayer. The plants were sprayed until runoff on all sides. Solvent treated control consists of 4 plants treated with diluent. Tests were held in a controlled environment holding room for three days at approximately 74° F. and 40° RH prior to grading. Evaluation was conducted by counting number of live aphids per plant. Number live count is entered into the database that calculates percent control using Abbott's correction formula. Abbott's Formula: (x−y)/x*100=percent corrected control where X=percent control living in check and Y=percent control living in treated group.

The following compounds gave 75% control of HELIZE at 50 μg/cm²: Compounds 1-16, 38, 40, 41, 43-45, 47

The following compounds gave 85% control of LAPHEG at 50 μg/cm²: Compounds 1-15, 17-20, 40, 41, 43, 46, 48-50

The following compounds gave 70% control of APHIGO at 200 ppm: Compounds 1, 3, 5, 7, 8, 13, 27.

### Acid & Salt Derivatives, and Solvates

The compounds disclosed in this invention can be in the form of pesticidally acceptable acid addition salts.

By way of non-limiting example, an amine function can form salts with hydrochloric, hydrobromic, sulfuric, phosphoric, acetic, benzoic, citric, malonic, salicylic, malic, fumaric, oxalic, succinic, tartaric, lactic, gluconic, ascorbic, maleic, aspartic, benzenesulfonic, methanesulfonic, ethanesulfonic, hydroxymethanesulfonic, and hydroxyethanesulfonic, acids.

Additionally, by way of non-limiting example, an acid function can form salts including those derived from alkali or alkaline earth metals and those derived from ammonia and amines. Examples of preferred cations include sodium, potassium, magnesium, and aminium cations.

The salts are prepared by contacting the free base form with a sufficient amount of the desired acid to produce a salt. The free base forms may be regenerated by treating the salt with a suitable dilute aqueous base solution such as dilute aqueous NaOH, potassium carbonate, ammonia, and sodium bicarbonate.

As an example, in many cases, a pesticide is modified to a more water soluble form e.g. 2,4-dichlorophenoxy acetic acid dimethyl amine salt is a more water soluble form of 2,4-dichlorophenoxy acetic acid a well known herbicide.

The compounds disclosed in this invention can also form stable complexes with solvent molecules that remain intact after the non-complexed solvent molecules are removed from the compounds. These complexes are often referred to as “solvates”.

### Stereoisomers

Certain compounds disclosed in this invention can exist as one or more stereoisomers. The various stereoisomers include geometric isomers, diastereomers, and enantiomers. Thus, the compounds disclosed in this invention include racemic mixtures, individual stereoisomers, and optically active mixtures.

It will be appreciated by those skilled in the art that one stereoisomer may be more active than the others. Individual stereoisomers and optically active mixtures may be obtained by selective synthetic procedures, by conventional synthetic procedures using resolved starting materials, or by conventional resolution procedures.

### Rests

In another embodiment, the invention disclosed in this document can be used to control pests.

In another embodiment, the invention disclosed in this document can be used to control pests of the Phylum Nematoda.

In another embodiment, the invention disclosed in this document can be used to control pests of the Phylum Arthropoda.

In another embodiment, the invention disclosed in this document can be used to control pests of the Subphylum Chelicerata.

In another embodiment, the invention disclosed in this document can be used to control pests of the Class Arachnida.

In another embodiment, the invention disclosed in this document can be used to control pests of the Subphylum Myriapoda.

In another embodiment, the invention disclosed in this document can be used to control pests of the Class Symphyla.

In another embodiment, the invention disclosed in this document can be used to control pests of the Subphylum Hexapoda.

In another embodiment, the invention disclosed in this document can be used to control pests of the Class Insecta.

In another embodiment, the invention disclosed in this document can be used to control Coleoptera (beetles). A non-exhaustive list of these pests includes, but is not limited to, Acanthoscelides spp. (weevils), Acanthoscelides obtectus (common bean weevil), Agrilus planipennis (emerald ash borer), Agriotes spp. (wireworms), Anoplophora glabripennis (Asian longhorned beetle), Anthonomus spp. (weevils), Anthonomus grandis (boll weevil), Aphidius spp., Apion spp. (weevils), Apogonia spp. (grubs), Ataenius spretulus (Black Turgrass Ataenius), Atomaria linearis (pygmy mangold beetle), Aulacophore spp., Bothynoderes punctiventris (beet root weevil), Bruchus spp. (weevils), Bruchus pisorum (pea weevil), Cacoesia spp., Callosobruchus maculatus (southern cow pea weevil), Carpophilus hemipteras (dried fruit beetle), Cassida vittata, Cerosterna spp, Cerotoma spp. (chrysomeids), Cerotoma trifurcata (bean leaf beetle), Ceutorhynchus spp. (weevils), Ceutorhynchus assimilis (cabbage seedpod weevil), Ceutorhynchus napi (cabbage curculio), Chaetocnema spp. (chrysomelids), Colaspis spp. (soil beetles), Conoderus scalaris, Conoderus stigmosus, Conotrachelus nenuphar (plum curculio), Cotinus nitidis (Green June beetle), Criceris asparagi (asparagus beetle), Cryptolestes ferrugineus (rusty grain beetle), Cryptolestes pusillus (flat grain beetle), Cryptolestes turcicus (Turkish grain beetle), Ctenicera spp. (wireworms), Curculio spp. (weevils), Cyclocephala spp., Cylindrocpturus adspersus (sunflower stem weevil), Deporaus marginatus (mango leaf-cutting weevil), Dernestes lardarius (larder beetle), Dermestes maculates (hide beetle), Diabrotica spp. (chrysolemids), Epilachna varivestis (Mexican bean beetle), Faustinus cubae, Hylobius pales (pales weevil), Hypera spp. (weevils), Hypera postica (alfalfa weevil), Hyperdoes spp. (Hyperodes weevil), Hypothenemus hampei (coffee berry beetle), Ips spp. (engravers), Lasioderma serricorne (cigarette beetle), Leptinotarsa decemlineata (Colorado potato beetle), Liogenys fuscus, Liogenys suturalis, Lissorhoptrus oryzophilus (rice water weevil), Lyctus spp. (wood beetles/powder post beetles), Maecolaspis joliveti, Megascelis spp., Melanotus communis, Meligethes spp., Meligethes aeneus (blossom beetle), Melolontha melolontha (common European cockchafer), Oberea brevis, Oberea linearis, Oryctes rhinoceros (date palm beetle), Oryzaephilus mercator (merchant grain beetle), Oryzaephilus surinamensis (sawtoothed grain beetle), Otiorhynchus spp. (weevils), Oulema melanopus (cereal leaf beetle), Oulema oryzae, Pantomorus spp. (weevils), Phyllophaga spp. (May/June beetle), Phyllophaga cuyabana, Phyllotreta spp. (chrysomelids), Phynchites spp., Popillia japonica (Japanese beetle), Prostephanus truncates (larger grain borer), Rhizopertha dominica (lesser grain borer), Rhizotrogus spp. (European chafer), Rhynchophorus spp. (weevils), Scolytus spp. (wood beetles), Shenophorus spp. (Billbug), Sitona lineatus (pea leaf weevil), Sitophilus spp. (grain weevils), Sitophilus granaries (granary weevil), Sitophilus oryzae (rice weevil), Stegobium paniceum (drugstore beetle), Tribolium spp. (flour beetles), Tribolium castaneum (red flour beetle), Tribolium confusum (confused flour beetle), Trogoderma variabile (warehouse beetle), and Zabrus tenebioides.

In another embodiment, the invention disclosed in this document can be used to control Dermaptera (earwigs).

In another embodiment, the invention disclosed in this document can be used to control Dictyoptera (cockroaches). A non-exhaustive list of these pests includes, but is not limited to, Blattella germanica (German cockroach), Blatta orientalis (oriental cockroach), Parcoblatta pennylvanica, Periplaneta americana (American cockroach), Periplaneta australoasiae (Australian cockroach), Periplaneta brunnea (brown cockroach), Periplaneta fuliginosa (smokybrown cockroach), Pyncoselus suninamensis (Surinam cockroach), and Supella longipalpa (brownbanded cockroach).

In another embodiment, the invention disclosed in this document can be used to control Diptera (true flies). A non-exhaustive list of these pests includes, but is not limited to, Aedes spp. (mosquitoes), Agromyza frontella (alfalfa blotch leafminer), Agromyza spp. (leaf miner flies), Anastrepha spp. (fruit flies), Anastrepha suspensa (Caribbean fruit fly), Anopheles spp. (mosquitoes), Batrocera spp. (fruit flies), Bactrocera cucurbitae (melon fly), Bactrocera dorsalis (oriental fruit fly), Ceratitis spp. (fruit flies), Ceratitis capitata (Mediterranea fruit fly), Chrysops spp. (deer flies), Cochliomyia spp. (screwworms), Contarinia spp. (Gall midges), Culex spp. (mosquitoes), Dasineura spp. (gall midges), Dasineura brassicae (cabbage gall midge), Delia spp., Delia platura (seedcorn maggot), Drosophila spp. (vinegar flies), Fannia spp. (filth flies), Fannia canicularis (little house fly), Fannia scalaris (latrine fly), Gasterophilus intestinalis (horse bot fly), Gracillia perseae, Haematobia irritans (horn fly), Hylemyia spp. (root maggots), Hypoderma lineatum (common cattle grub), Liriomyza spp. (leafminer flies), Liriomyza brassica (serpentine leafminer), Melophagus ovinus (sheep ked), Musca spp. (muscid flies), Musca autumnalis (face fly), Musca domestica (house fly), Oestrus ovis (sheep bot fly), Oscinella frit (frit fly), Pegomyia betae (beet leafminer), Phorbia spp., Psila rosae (carrot rust fly), Rhagoletis cerasi (cherry fruit fly), Rhagoletis pomonella (apple maggot), Sitodiplosis mosellana (orange wheat blossom midge), Stomoxys calcitrans (stable fly), Tabanus spp. (horse flies), and Tipula spp. (crane flies).

In another embodiment, the invention disclosed in this document can be used to control Hemiptera (true bugs). A non-exhaustive list of these pests includes, but is not limited to, Acrosternum hilare (green stink bug), Blissus leucopterus (chinch bug), Calocoris norvegicus (potato mirid), Cimex hemipterus (tropical bed bug), Cimex lectularius (bed bug), Dagbertus fasciatus, Dichelops furcatus, Dysdercus suturellus (cotton stainer), Edessa meditabunda, Eurygaster maura (cereal bug), Euschistus heros, Euschistus servus (brown stink bug), Helopeltis antonii, Helopeltis theivora (tea blight plantbug), Lagynotomus spp. (stink bugs), Leptocorisa oratorius, Leptocorisa varicornis, Lygus spp. (plant bugs), Lygus hesperus (western tarnished plant bug), Maconellicoccus hirsutus, Neurocolpus longirostris, Nezara viridula (southern green stink bug), Phytocoris spp. (plant bugs), Phytocoris californicus, Phytocoris relativus, Piezodorus guildingi, Poecilocapsus lineatus (fourlined plant bug), Psallus vaccinicola, Pseudacysta perseae, Scaptocoris castanea, and Triatoma spp. (bloodsucking conenose bugs/kissing bugs).

In another embodiment, the invention disclosed in this document can be used to control Homoptera (aphids, scales, whiteflies, leafhoppers). A non-exhaustive list of these pests includes, but is not limited to, Acrythosiphon pisum (pea aphid), Adelges spp. (adelgids), Aleurodes proletella (cabbage whitefly), Aleurodicus disperses, Aleurothrixus floccosus (woolly whitefly), Aluacaspis spp., Amrasca bigutella bigutella, Aphrophora spp. (leafhoppers), Aonidiella aurantii (California red scale), Aphis spp. (aphids), Aphis gossypii (cotton aphid), Aphis pomi (apple aphid), Aulacorthum solani (foxglove aphid), Bemisia spp. (whiteflies), Bemisia argentifolii, Bemisia tabaci (sweetpotato whitefly), Brachycolus noxius (Russian aphid), Brachycorynella asparagi (asparagus aphid), Brevennia rehi, Brevicoryne brassicae (cabbage aphid), Ceroplastes spp. (scales), Ceroplastes rubens (red wax scale), Chionaspis spp. (scales), Chrysomphalus spp. (scales), Coccus spp. (scales), Dysaphis plantaginea (rosy apple aphid), Empoasca spp. (leafhoppers), Eriosoma lanigerum (woolly apple aphid), Icerya purchasi (cottony cushion scale), Idioscopus nitidulus (mango leafhopper), Laodelphax striatellus (smaller brown planthopper), Lepidosaphes spp., Macrosiphum spp., Macrosiphum euphorbiae (potato aphid), Macrosiphum granarium (English grain aphid), Macrosiphum rosae (rose aphid), Macrosteles quadrilineatus (aster leafhopper), Mahanarva frimbiolata, Metopolophium dirhodum (rose grain aphid), Mictis longicornis, Myzus persicae (green peach aphid), Nephotettix spp. (leafhoppers), Nephotettix cinctipes (green leafhopper), Nilaparvata lugens (brown planthopper), Parlatoria pergandii (chaff scale), Parlatoria ziziphi (ebony scale), Peregrinus maidis (corn delphacid), Philaenus spp. (spittlebugs), Phylloxera vitifoliae (grape phylloxera), Physokermes piceae (spruce bud scale), Planococcus spp. (mealybugs), Pseudococcus spp. (mealybugs), Pseudococcus brevipes (pine apple mealybug), Quadraspidiotus perniciosus (San Jose scale), Rhapalosiphum spp. (aphids), Rhapalosiphum maida (corn leaf aphid), Rhapalosiphum padi (oat bird-cherry aphid), Saissetia spp. (scales), Saissetia oleae (black scale), Schizaphis graminum (greenbug), Sitobion avenae (English grain aphid), Sogatella furcifera (white-backed planthopper), Therioaphis spp. (aphids), Toumeyella spp. (scales), Toxoptera spp. (aphids), Trialeurodes spp. (whiteflies), Trialeurodes vaporariorum (greenhouse whitefly), Trialeurodes abutiloneus (bandedwing whitefly), Unaspis spp. (scales), Unaspis yanonensis (arrowhead scale), and Zulia entreriana.

In another embodiment, the invention disclosed in this document can be used to control Hymenoptera (ants, wasps, and bees). A non-exhaustive list of these pests includes, but is not limited to, Acromyrrmex spp., Athalia rosae, Atta spp. (leafcutting ants), Camponotus spp. (carpenter ants), Diprion spp. (sawflies), Formica spp. (ants), Iridomyrmex humilis (Argentine ant), Monomorium ssp., Monomorium minumum (little black ant), Monomorium pharaonis (Pharaoh ant), Neodiprion spp. (sawflies), Pogonomyrmex spp. (harvester ants), Polistes spp. (paper wasps), Solenopsis spp. (fire ants), Tapoinoma sessile (odorous house ant), Tetranomorium spp. (pavement ants), Vespula spp. (yellow jackets), and Xylocopa spp. (carpenter bees).

In another embodiment, the invention disclosed in this document can be used to control Isoptera (termites). A non-exhaustive list of these pests includes, but is not limited to, Coptotermes spp., Coptotermes curvignathus, Coptotermes frenchii, Coptotermes formosanus (Formosan subterranean termite), Corniternes spp. (nasute termites), Cryptotermes spp. (drywood termites), Heterotermes spp. (desert subterranean termites), Heterotermes aureus, Kalotermes spp. (drywood termites), Incistitermes spp. (drywood termites), Macrotermes spp. (fungus growing termites), Marginitermes spp. (drywood termites), Microcerotermes spp. (harvester termites), Microtermes obesi, Procornitermes spp., Reticulitermes spp. (subterranean termites), Reticulitermes banyulensis, Reticulitermes grassei, Reticulitermes flavipes (eastern subterranean termite), Reticulitermes hageni, Reticulitermes hesperus (western subterranean termite), Reticulitermes santonensis, Reticulitermes speratus, Reticulitermes tibialis, Reticulitermes virginicus, Schedorhinotermes spp., and Zootermopsis spp. (rotten-wood termites).

In another embodiment, the invention disclosed in this document can be used to control Lepidoptera (moths and butterflies). A non-exhaustive list of these pests includes, but is not limited to, Achoea janata, Adoxophyes spp., Adoxophyes orana, Agrotis spp. (cutworms), Agrotis ipsilon (black cutworm), Alabama argillacea (cotton leafworm), Amorbia cuneana, Amyelosis transitella (navel orangeworm), Anacamptodes defectaria, Anarsia lineatella (peach twig borer), Anomis sabulifera (jute looper), Anticarsia gemmatalis (velvetbean caterpillar), Archips argyrospila (fruittree leafroller), Archips rosana (rose leaf roller), Argyrotaenia spp. (tortricid moths), Argyrotaenia citrana (orange tortrix), Autographa gamma, Bonagota cranaodes, Borbo cinnara (rice leaf folder), Bucculatrix thurberiella (cotton leafperforator), Caloptilia spp. (leaf miners), Capua reticulana, Carposina niponensis (peach fruit moth), Chilo spp., Chlumetia transversa (mango shoot borer), Choristoneura rosaceana (obliquebanded leafroller), Chrysodeixis spp., Cnaphalocerus medinalis (grass leafroller), Colias spp., Conpomorpha cramerella, Cossus cossus (carpenter moth), Crambus spp. (Sod webworms), Cydia funebrana (plum fruit moth), Cydia molesta (oriental fruit moth), Cydia nignicana (pea moth), Cydia pomonella (codling moth), Darna diducta, Diaphania spp. (stem borers), Diatraea spp. (stalk borers), Diatraea saccharalis (sugarcane borer), Diatraea graniosella (southwester corn borer), Earias spp. (bollworms), Earias insulata (Egyptian bollworm), Earias vitella (rough northern bollworm), Ecdytopopha aurantianum, Elasmopalpus lignosellus (lesser cornstalk borer), Epiphysias postruttana (light brown apple moth), Ephestia spp. (flour moths), Ephestia cautella (almond moth), Ephestia elutella (tobbaco moth), Ephestia kuehniella (Mediterranean flour moth), Epimeces spp., Epinotia aporema, Erionota thrax (banana skipper), Eupoecilia ambiguella (grape berry moth), Euxoa auxiliaris (army cutworm), Feltia spp. (cutworms), Gortyna spp. (stemborers), Grapholita molesta (oriental fruit moth), Hedylepta indicata (bean leaf webber), Helicoverpa spp. (noctuid moths), Helicoverpa annigera (cotton bollworm), Helicoverpa zea (bollworm/corn earworm), Heliothis spp. (noctuid moths), Heliothis virescens (tobacco budworm), Hellula undalis (cabbage webworm), Indarbela spp. (root borers), Keiferia lycopersicella (tomato pinworm), Leucinodes orbonalis (eggplant fruit borer), Leucoptera malifoliella, Lithocollectis spp., Lobesia botrana (grape fruit moth), Loxagrotis spp. (noctuid moths), Loxagrotis albicosta (western bean cutworm), Lymantria dispar (gypsy moth), Lyonetia clerkella (apple leaf miner), Mahasena corbetti (oil palm bagworm), Malacosoma spp. (tent caterpillars), Mamestra brassicae (cabbage armyworm), Maruca testulalis (bean pod borer), Metisa plana (bagworm), Mythimna unipuncta (true armyworm), Neoleucinodes elegantalis (small tomato borer), Nymphula depunctalis (rice caseworm), Operophthera brumata (winter moth), Ostrinia nubilalis (European corn borer), Oxydia vesulia, Pandemis cerasana (common currant tortrix), Pandemis heparana (brown apple tortrix), Papilio demodocus, Pectinophora gossypiella (pink bollworm), Peridroma spp. (cutworms), Peridroma saucia (variegated cutworm), Perileucoptera coffeella (white coffee leafminer), Phthorimaea operculella (potato tuber moth), Phyllocnisitis citrella, Phyllonorycter spp. (leafminers), Pieris rapae (imported cabbageworm), Plathypena scabra, Plodia interpunctella (Indian meal moth), Plutella xylostella (diamondback moth), Polychrosis viteana (grape berry moth), Prays endocarpa, Prays oleae (olive moth), Pseudaletia spp. (noctuid moths), Pseudaletia unipunctata (armyworm), Pseudoplusia includens (soybean looper), Rachiplusia nu, Scirpophaga incertulas, Sesamia spp. (stemborers), Sesamia inferens (pink rice stem borer), Sesamia nonagrioides, Setora nitens, Sitotroga cerealella (Angoumois grain moth), Sparganothis pilleriana, Spodoptera spp. (armyworms), Spodoptera exigua (beet armyworm), Spodoptera fugiperda (fall armyworm), Spodoptera oridania (southern armyworm), Synanthedon spp. (root borers), Thecla basilides, Thernisia gemmatalis, Tineola bisselliella (webbing clothes moth), Trichoplusia ni (cabbage looper), Tuta absoluta, Yponomeuta spp., Zeuzera coffeae (red branch borer), and Zeuzera pyrina (leopard moth).

In another embodiment, the invention disclosed in this document can be used to control Mallophaga (chewing lice). A non-exhaustive list of these pests includes, but is not limited to, Bovicola ovis (sheep biting louse), Menacanthus stramineus (chicken body louse), and Menopon gallinea (common hen house).

In another embodiment, the invention disclosed in this document can be used to control Orthoptera (grasshoppers, locusts, and crickets). A non-exhaustive list of these pests includes, but is not limited to, Anabrus simplex (Mormon cricket), Gryllotalpidae (mole crickets), Locusta migratoria, Melanoplus spp. (grasshoppers), Microcentrum retinerve (angularwinged katydid), Pterophylla spp. (kaydids), chistocerca gregaria, Scudderia furcata (forktailed bush katydid), and Valanga nigricorni.

In another embodiment, the invention disclosed in this document can be used to control Phthiraptera (sucking lice). A non-exhaustive list of these pests includes, but is not limited to, Haematopinus spp. (cattle and hog lice), Linognathus ovillus (sheep louse), Pediculus humanus capitis (human body louse), Pediculus humanus humanus (human body lice), and Pthirus pubis (crab louse),

In another embodiment, the invention disclosed in this document can be used to control Siphonaptera (fleas). A non-exhaustive list of these pests includes, but is not limited to, Ctenocephalides canis (dog flea), Ctenocephalides felis (cat flea), and Pulex irritans (human flea).

In another embodiment, the invention disclosed in this document can be used to control Thysanoptera (thrips). A non-exhaustive list of these pests includes, but is not limited to, Frankliniella fusca (tobacco thrips), Frankliniella occidentalis (western flower thrips), Frankliniella shultzei Frankliniella williamsi (corn thrips), Heliothrips haemorrhaidalis (greenhouse thrips), Riphiphorothrips cruentatus, Scirtothrips spp., Scirtothrips citri (citrus thrips), Scirtothrips dorsalis (yellow tea thrips), Taeniothrips rhopalantennalis, and Thrips spp.

In another embodiment, the invention disclosed in this document can be used to control Thysanura (bristletails). A non-exhaustive list of these pests includes, but is not limited to, Lepisma spp. (silverfish) and Thermobia spp. (firebrats).

In another embodiment, the invention disclosed in this document can be used to control Acarina (mites and ticks). A non-exhaustive list of these pests includes, but is not limited to, Acarapsis woodi (tracheal mite of honeybees), Acarus spp. (food mites), Acarus siro (grain mite), Aceria mangiferae (mango bud mite), Aculops spp., Aculops lycopersici (tomato russet mite), Aculops pelekasi, Aculus pelekassi, Aculus schlechtendali (apple rust mite), Amblyomma americanum (lone star tick), Boophilus spp. (ticks), Brevipalpus obovatus (privet mite), Brevipalpus phoenicis (red and black flat mite), Demodex spp. (mange mites), Dernacentor spp. (hard ticks), Dermacentor variabilis (american dog tick), Dennatophagoides pteronyssinus (house dust mite), Eotetranycus spp., Eotetranychus carpini (yellow spider mite), Epitimerus spp., Eriophyes spp., Ixodes spp. (ticks), Metatetranycus spp., Notoedres cati, Oligonychus spp., Oligonychus coffee, Oligonychus ilicus (southern red mite), Panonychus spp., Panonychus citri (citrus red mite), Panonychus ulmi (European red mite), Phyllocoptruta oleivora (citrus rust mite), Polyphagotarsonemun latus (broad mite), Rhipicephalus sanguineus (brown dog tick), Rhizoglyphus spp. (bulb mites), Sarcoptes scabiei (itch mite), Tegolophus perseaflorae, Tetranychus spp., Tetranychus urticae (twospotted spider mite), and Varroa destructor (honey bee mite).

In another embodiment, the invention disclosed in this document can be used to control Nematoda (nematodes). A non-exhaustive list of these pests includes, but is not limited to, Aphelenchoides spp. (bud and leaf & pine wood nematodes), Belonolaimus spp. (sting nematodes), Criconemella spp. (ring nematodes), Dirofilaria immitis (dog heartwom), Ditylenchus spp. (stem and bulb nematodes), Heterodera spp. (cyst nematodes), Heterodera zeae (corn cyst nematode), Hirschmanniella spp. (root nematodes), Hoplolaimus spp. (lance nematodes), Meloidogyne spp. (root knot nematodes), Meloidogyne incognita (root knot nematode), Onchocerca volvulus (hook-tail worm), Pratylenchus spp. (lesion nematodes), Radopholus spp. (burrowing nematodes), and Rotylenchus reniformis (kidney-shaped nematode).

In another embodiment, the invention disclosed in this document can be used to control Symphyla (symphylans). A non-exhaustive list of these pests includes, but is not limited to, Scutigerella immaculata.

For more detailed information consult “Handbook of Pest Control—The Behavior, Life Histroy, and Control of Household Pests” by Arnold Mallis, 9^{th} Edition, copyright 2004 by GIE Media Inc.

### Mixtures

Some of the pesticides that can be employed beneficially in combination with the invention disclosed in this document include, but are not limited to the following:

1,2 dichloropropane, 1,3 dichloropropene,

abamectin, acephate, acequinocyl, acetamiprid, acethion, acetoprole, acrinathrin, acrylonitrile, alanycarb, aldicarb, aldoxycarb, aldrin, allethrin, allosamidin, allyxycarb, alpha cypermethrin, alpha ecdysone, amidithion, amidoflumet, aminocarb, amiton, amitraz, anabasine, arsenous oxide, athidathion, azadirachtin, azamethiphos, azinphos ethyl, azinphos methyl, azobenzene, azocyclotin, azothoate,

barium hexafluorosilicate, barthrin, benclothiaz, bendiocarb, benfuracarb, benomyl, benoxafos, bensultap, benzoximate, benzyl benzoate, beta cyfluthrin, beta cypermethrin, bifenazate, bifenthrin, binapacryl, bioallethrin, bioethanomethrin, biopermethrin, bistrifluoron, borax, boric acid, bromfenvinfos, bromo DDT, bromocyclen, bromophos, bromophos ethyl, bromopropylate, bufencarb, buprofezin, butacarb, butathiofos, butocarboxim, butonate, butoxycarboxim,

cadusafos, calcium arsenate, calcium polysulfide, camphechlor, carbanolate, carbaryl, carbofuran, carbon disulfide, carbon tetrachloride, carbophenothion, carbosulfan, cartap, chinomethionat, chlorantraniliprole, chlorbenside, chlorbicyclen, chlordane, chlordecone, chlordimeform, chlorethoxyfos, chlorfenapyr, chlorfenethol, chlorfenson, chlorfensulphide, chlorfenvinphos, chlorfluazuron, chlormephos, chlorobenzilate, chloroform, chloromebuform, chloromethiuron, chloropicrin, chloropropylate, chlorphoxim, chlorprazophos, chlorpyrifos, chlorpyrifos methyl, chlorthiophos, chromafenozide, cinerin I, cinerin II, cismethrin, cloethocarb, clofentezine, closantel, clothianidin, copper acetoarsenite, copper arsenate, copper naphthenate, copper oleate, coumaphos, coumithoate, crotamiton, crotoxyphos, crufomate, cryolite, cyanofenphos, cyanophos, cyanthoate, cyclethrin, cycloprothrin, cyenopyrafen, cyflumetofen, cyfluthrin, cyhalothrin, cyhexatin, cypermethrin, cyphenothrin, cyromazine, cythioate,

d-limonene, dazomet, DBCP, DCIP, DDT, decarbofuran, deltamethrin, demephion, demephion O, demephion S, demeton, demeton methyl, demeton O, demeton O methyl, demeton S, demeton S methyl, demeton S methylsulphon, diafenthiuron, dialifos, diamidafos, diazinon, dicapthon, dichlofenthion, dichlofluanid, dichlorvos, dicofol, dicresyl, dicrotophos, dicyclanil, dieldrin, dienochlor, diflovidazin, diflubenzuron, dilor, dimefluthrin, dimefox, dimetan, dimethoate, dimethrin, dimethylvinphos, dimetilan, dinex, dinobuton, dinocap, dinocap 4, dinocap 6, dinocton, dinopenton, dinoprop, dinosam, dinosulfon, dinotefuran, dinoterbon, diofenolan, dioxabenzofos, dioxacarb, dioxathion, diphenyl sulfone, disulfuram, disulfoton, dithicrofos, DNOC, dofenapyn, doramectin,

ecdysterone, emamectin, EMPC, empenthrin, endosulfan, endothion, endrin, EPN, epofenonane, eprinomectin, esfenvalerate, etaphos, ethiofencarb, ethion, ethiprole, ethoate methyl, ethoprophos, ethyl DDD, ethyl formate, ethylene dibromide, ethylene dichloride, ethylene oxide, etofenprox, etoxazole, etrimfos, EXD,

famphur, fenamiphos, fenazaflor, fenazaquin, fenbutatin oxide, fenchlorphos, fenethacarb, fenfluthrin, fenitrothion, fenobucarb, fenothiocarb, fenoxacrim, fenoxycarb, fenpirithrin, fenpropathrin, fenpyroximate, fenson, fensulfothion, fenthion, fenthion ethyl, fentrifanil, fenvalerate, fipronil, flonicamid, fluacrypyrim, fluazuron, flubendiamide, flubenzimine, flucofuron, flucycloxuron, flucythrinate, fluenetil, flufenerim, flufenoxuron, flufenprox, flumethrin, fluorbenside, fluvalinate, fonofos, formetanate, formothion, formparanate, fosmethilan, fospirate, fosthiazate, fosthietan, fosthietan, furathiocarb, furethrin, furfural,

gamma cyhalothrin, gamma HCH,

halfenprox, halofenozide, HCH, HEOD, heptachlor, heptenophos, heterophos, hexaflumuron, hexythiazox, HHDN, hydramethylnon, hydrogen cyanide, hydroprene, hyquincarb,

imicyafos, imidacloprid, imiprothrin, indoxacarb, iodomethane, IPSP, isamidofos, isazofos, isobenzan, isocarbophos, isodrin, isofenphos, isoprocarb, isoprothiolane, isothioate, isoxathion, ivermectin

jasmolin I, jasmolin II, jodfenphos, juvenile hormone I, juvenile hormone II, juvenile hormone III, kelevan, kinoprene,

lambda cyhalothrin, lead arsenate, lepimectin, leptophos, lindane, lirimfos, lufenuron, lythidathion,

malathion, malonoben, mazidox, mecarbam, mecarphon, menazon, mephosfolan, mercurous chloride, mesulfen, mesulfenfos, metaflumizone, metam, methacrifos, methamidophos, methidathion, methiocarb, methocrotophos, methomyl, methoprene, methoxychlor, methoxyfenozide, methyl bromide, methyl isothiocyanate, methylchloroform, methylene chloride, metofluthrin, metolcarb, metoxadiazone, mevinphos, mexacarbate, milbemectin, milbemycin oxime, mipafox, mirex, MNAF, monocrotophos, morphothion, moxidectin,

naftalofos, naled, naphthalene, nicotine, nifluridide, nikkomycins, nitenpyram, nithiazine, nitrilacarb, novaluron, noviflumuron,

omethoate, oxamyl, oxydemeton methyl, oxydeprofos, oxydisulfoton,

paradichlorobenzene, parathion, parathion methyl, penfluoron, pentachlorophenol, permethrin, phenkapton, phenothrin, phenthoate, phorate, phosalone, phosfolan, phosmet, phosnichlor, phosphamidon, phosphine, phosphocarb, phoxim, phoxim methyl, pirimetaphos, pirimicarb, pirimiphos ethyl, pirimiphos methyl, potassium arsenite, potassium thiocyanate, pp′ DDT, prallethrin, precocene I, precocene II, precocene III, primidophos, proclonol, profenofos, profluthrin, promacyl, promecarb, propaphos, propargite, propetamphos, propoxur, prothidathion, prothiofos, prothoate, protrifenbute, pyraclofos, pyrafluprole, pyrazophos, pyresmethrin, pyrethrin I, pyrethrin II, pyridaben, pyridalyl, pyridaphenthion, pyrifluquinazon, pyrimidifen, pyrimitate, pyriprole, pyriproxyfen,

quassia, quinalphos, quinalphos, quinalphos methyl, quinothion, quantifies,

rafoxanide, resmethrin, rotenone, ryania,

sabadilla, schradan, selamectin, silafluofen, sodium arsenite, sodium fluoride, sodium hexafluorosilicate, sodium thiocyanate, sophamide, spinetoram, spinosad, spirodiclofen, spiromesifen, spirotetramat, sulcofuron, sulfuram, sulflurarnid, sulfotep, sulfur, sulfuryl fluoride, sulprofos,

tau fluvalinate, tazimcarb, TDE, tebufenozide, tebufenpyrad, tebupirimfos, teflubenzuron, tefluthrin, temephos, TEPP, terallethrin, terbufos, tetrachloroethane, tetrachlorvinphos, tetradifon, tetramethrin, tetranactin, tetrasul, theta cypermethrin, thiacloprid, thiamethoxam, thicrofos, thiocarboxime, thiocyclam, thiodicarb, thiofanox, thiometon, thionazin, thioquinox, thiosultap, thuringiensin, tolfenpyrad, tralomethrin, transfluthrin, transpermethrin, triarathene, triazamate, triazophos, trichlorfon, trichlormetaphos 3, trichloronat, trifenofos, triflumuron, trimethacarb, triprene,

vamidothion, vamidothion, vaniliprole, vaniliprole, XMC, xylylcarb,

zeta cypermethrin and zolaprofos.

Additionally, any combination of the above pesticides can be used.

The invention disclosed in this document can also be used with herbicides and fungicides, both for reasons of economy and synergy.

The invention disclosed in this document can be used with antimicrobials, bactericides, defoliants, safeners, synergists, algaecides, attractants, desiccants, pheromones, repellants, animal dips, avicides, disinfectants, semiochemicals, and molluscicides (these categories not necessarily mutually exclusive) for reasons of economy, and synergy.

For more information consult “Compendium of Pesticide Common Names” located at http://www.alanwood.net/pesticides/index.html as of the filing date of this document. Also consult “The Pesticide Manual” 14^{th} Edition, edited by C D S Tomlin, copyright 2006 by British Crop Production Council.

### Synergistic Mixtures

The invention disclosed in this document can be used with other compounds such as the ones mentioned under the heading “Mixtures” to form synergistic mixtures where the mode of action of the compounds in the mixtures are the same, similar, or different.

Examples of mode of actions include, but are not limited to: acetyl choline esterase inhibitor; sodium channel modulator; chitin biosynthesis inhibitor; GABA-gated chloride channel antagonist; GABA and glutamate-gated chloride channel agonist; acetyl choline receptor agonist; MET I inhibitor; Mg-stimulated ATPase inhibitor; nicotinic acetylcholine receptor; Midgut membrane disrupter; and oxidative phosphorylation disrupter.

Additionally, the following compounds are known as synergists and can be used with the invention disclosed in this document: piperonyl butoxide, piprotal, propyl isome, sesamex, sesamolin, and sulfoxide.

### Formulations

A pesticide is rarely suitable for application in its pure form. It is usually necessary to add other substances so that the pesticide can be used at the required concentration and in an appropriate form, permitting ease of application, handling, transportation, storage, and maximum pesticide activity. Thus, pesticides are formulated into, for example, baits, concentrated emulsions, dusts, emulsifiable concentrates, fumigants, gels, granules, microencapsulations, seed treatments, suspension concentrates, suspoemulsions, tablets, water soluble liquids, water dispersible granules or dry flowables, wettable powders, and ultra low volume solutions.

For further information on formulation types see “Catalogue of pesticide formulation types and international coding system” Technical Monograph n^{o} 2, 5^{th} Edition by CropLife International (2002).

Pesticides are applied most often as aqueous suspensions or emulsions prepared from concentrated formulations of such pesticides. Such water-soluble, water-suspendable, or emulsifiable formulations, are either solids, usually known as wettable powders, or water dispersible granules, or liquids usually known as emulsifiable concentrates, or aqueous suspensions. Wettable powders, which may be compacted to form water dispersible granules, comprise an intimate mixture of the pesticide, a carrier, and surfactants. The concentration of the pesticide is usually from about 10% to about 90% by weight. The carrier is usually chosen from among the attapulgite clays, the montmorillonite clays, the diatomaceous earths, or the purified silicates. Effective surfactants, comprising from about 0.5% to about 10% of the wettable powder, are found among sulfonated lignins, condensed naphthalenesulfonates, naphthalenesulfonates, alkylbenzenesulfonates, alkyl sulfates, and nonionic surfactants such as ethylene oxide adducts of alkyl phenols.

Emulsifiable concentrates of pesticides comprise a convenient concentration of a pesticide, such as from about 50 to about 500 grams per liter of liquid dissolved in a carrier that is either a water miscible solvent or a mixture of water-immiscible organic solvent and emulsifiers. Useful organic solvents include aromatics, especially xylenes and petroleum fractions, especially the high-boiling naphthalenic and olefinic portions of petroleum such as heavy aromatic naphtha. Other organic solvents may also be used, such as the terpenic solvents including rosin derivatives, aliphatic ketones such as cyclohexanone, and complex alcohols such as 2-ethoxyethanol. Suitable emulsifiers for emulsifiable concentrates are chosen from conventional anionic and nonionic surfactants.

Aqueous suspensions comprise suspensions of water-insoluble pesticides dispersed in an aqueous carrier at a concentration in the range from about 5% to about 50% by weight. Suspensions are prepared by finely grinding the pesticide and vigorously mixing it into a carrier comprised of water and surfactants. Ingredients, such as inorganic salts and synthetic or natural gums, may also be added, to increase the density and viscosity of the aqueous carrier. It is often most effective to grind and mix the pesticide at the same time by preparing the aqueous mixture and homogenizing it in an implement such as a sand mill, ball mill, or piston-type homogenizer.

Pesticides may also be applied as granular compositions that are particularly useful for applications to the soil. Granular compositions usually contain from about 0.5% to about 10% by weight of the pesticide, dispersed in a carrier that comprises clay or a similar substance. Such compositions are usually prepared by dissolving the pesticide in a suitable solvent and applying it to a granular carrier which has been pre-formed to the appropriate particle size, in the range of from about 0.5 to 3 mm. Such compositions may also be formulated by making a dough or paste of the carrier and compound and crushing and drying to obtain the desired granular particle size.

Dusts containing a pesticide are prepared by intimately mixing the pesticide in powdered form with a suitable dusty agricultural carrier, such as kaolin clay, ground volcanic rock, and the like. Dusts can suitably contain from about 1% to about 10% of the pesticide. They can be applied as a seed dressing, or as a foliage application with a dust blower machine.

It is equally practical to apply a pesticide in the form of a solution in an appropriate organic solvent, usually petroleum oil, such as the spray oils, which are widely used in agricultural chemistry.

Pesticides can also be applied in the form of an aerosol composition. In such compositions the pesticide is dissolved or dispersed in a carrier, which is a pressure-generating propellant mixture. The aerosol composition is packaged in a container from which the mixture is dispensed through an atomizing valve.

Pesticide baits are formed when the pesticide is mixed with food or an attractant or both. When the pests eat the bait they also consume the pesticide. Baits may take the form of granules, gels, flowable powders, liquids, or solids. They are use in pest harborages.

Fumigants are pesticides that have a relatively high vapor pressure and hence can exist as a gas in sufficient concentrations to kill pests in soil or enclosed spaces. The toxicity of the fumigant is proportional to its concentration and the exposure time. They are characterized by a good capacity for diffusion and act by penetrating the pest's respiratory system or being absorbed through the pest's cuticle. Fumigants are applied to control stored product pests under gas proof sheets, in gas sealed rooms or buildings or in special chambers.

Pesticides can be microencapsulated by suspending the pesticide particles or droplets in plastic polymers of various types. By altering the chemistry of the polymer or by changing factors in the processing, microcapsules can be formed of various sizes, solubility, wall thicknesses, and degrees of penetrability. These factors govern the speed with which the active ingredient within is released, which, in turn, affects the residual performance, speed of action, and odor of the product.

Oil solution concentrates are made by dissolving pesticide in a solvent that will hold the pesticide in solution. Oil solutions of a pesticide usually provide faster knockdown and kill of pests than other formulations due to the solvents themselves having pesticidal action and the dissolution of the waxy covering of the integument increasing the speed of uptake of the pesticide. Other advantages of oil solutions include better storage stability, better penetration of crevices, and better adhesion to greasy surfaces.

Another embodiment is an oil-in-water emulsion, wherein the emulsion comprises oily globules which are each provided with a lamellar liquid crystal coating and are dispersed in an aqueous phase, wherein each oily globule comprises at least one compound which is agriculturally active, and is individually coated with a monolamellar or oligolamellar layer comprising: (1) at least one non-ionic lipophilic surface-active agent, (2) at least one non-ionic hydrophilic surface-active agent and (3) at least one ionic surface-active agent, wherein the globules having a mean particle diameter of less than 800 nanometers. Further information on the embodiment is disclosed in U.S. patent publication 20070027034 published Feb. 1, 2007, having patent application Ser. No. 11/495,228. For ease of use this embodiment will be referred to as “OIWE”.

For further information consult “Insect Pest Management” 2^{nd} Edition by D. Dent, copyright CAB International (2000). Additionally, for more detailed information consult “Handbook of Pest Control—The Behavior, Life Histroy, and Control of Household Pests” by Arnold Mallis, 9^{th} Edition, copyright 2004 by GIE Media Inc.

### Other Formulation Components

Generally, the invention disclosed in this document when used in a formulation, such formulation can also contain other components. These components include, but are not limited to, (this is a non-exhaustive and non-mutually exclusive list) wetters, spreaders, stickers, penetrants, buffers, sequestering agents, drift reduction agents, compatibility agents, anti-foam agents, cleaning agents, and emulsifiers. A few components are described forthwith.

A wetting agent is a substance that when added to a liquid increases the spreading or penetration power of the liquid by reducing the interfacial tension between the liquid and the surface on which it is spreading. Wetting agents are used for two main functions in agrochemical formulations: during processing and manufacture to increase the rate of wetting of powders in water to make concentrates for soluble liquids or suspension concentrates; and during mixing of a product with water in a spray tank to reduce the wetting time of wettable powders and to improve the penetration of water into water-dispersible granules. Examples of wetting agents used in wettable powder, suspension concentrate, and water-dispersible granule formulations are: sodium lauryl sulphate; sodium dioctyl sulphosuccinate; alkyl phenol ethoxylates; and aliphatic alcohol ethoxylates.

A dispersing agent is a substance which adsorbs onto the surface of a particles and helps to preserve the state of dispersion of the particles and prevents them from reaggregating. Dispersing agents are added to agrochemical formulations to facilitate dispersion and suspension during manufacture, and to ensure the particles redisperse into water in a spray tank. They are widely used in wettable powders, suspension concentrates and water-dispersible granules. Surfactants that are used as dispersing agents have the ability to adsorb strongly onto a particle surface and provide a charged or steric barrier to reaggregation of particles. The most commonly used surfactants are anionic, non-ionic, or mixtures of the two types. For wettable powder formulations, the most common dispersing agents are sodium lignosulphonates. For suspension concentrates, very good adsorption and stabilization are obtained using polyelectrolytes, such as sodium naphthalene sulphonate formaldehyde condensates. Tristyrylphenol ethoxylate phosphate esters are also used. Non-ionics such as alkylarylethylene oxide condensates and EO-PO block copolymers are sometimes combined with anionics as dispersing agents for suspension concentrates. In recent years, new types of very high molecular weight polymeric surfactants have been developed as dispersing agents. These have very long hydrophobic ‘backbones’ and a large number of ethylene oxide chains forming the ‘teeth’ of a ‘comb’ surfactant. These high molecular weight polymers can give very good long-term stability to suspension concentrates because the hydrophobic backbones have many anchoring points onto the particle surfaces. Examples of dispersing agents used in agrochemical formulations are: sodium lignosulphonates; sodium naphthalene sulphonate formaldehyde condensates; Tristyrylphenol ethoxylate phosphate esters; aliphatic alcohol ethoxylates; alky ethoxylates; EO-PO block copolymers; and graft copolymers.

An emulsifying agent is a substance which stabilizes a suspension of droplets of one liquid phase in another liquid phase. Without the emulsifying agent the two liquids would separate into two immiscible liquid phases. The most commonly used emulsifier blends contain alkylphenol or aliphatic alcohol with 12 or more ethylene oxide units and the oil-soluble calcium salt of dodecylbenzene sulphonic acid. A range of hydrophile-lipophile balance (“HLB”) values from 8 to 18 will normally provide good stable emulsions. Emulsion stability can sometimes be improved by the addition of a small amount of an EO-PO block copolymer surfactant.

A solubilizing agent is a surfactant which will form micelles in water at concentrations above the critical micelle concentration. The micelles are then able to dissolve or solubilized water-insoluble materials inside the hydrophobic part of the micelle. The type of surfactants usually used for solubilization are non-ionics: sorbitan monooleates; sorbitan monooleate ethoxylates; and methyl oleate esters.

Surfactants are sometimes used, either alone or with other additives such as mineral or vegetable oils as adjuvants to spray-tank mixes to improve the biological performance of the pesticide on the target. The types of surfactants used for bioenhancement depend generally on the nature and mode of action of the pesticide. However, they are often non-ionics such as: alky ethoxylates; linear aliphatic alcohol ethoxylates; aliphatic amine ethoxylates.

A carrier or diluent in an agricultural formulation is a material added to the pesticide to give a product of the required strength. Carriers are usually materials with high absorptive capacities, while diluents are usually materials with low absorptive capacities. Carriers and diluents are used in the formulation of dusts, wettable powders, granules and water-dispersible granules.

Organic solvents are used mainly in the formulation of emulsifiable concentrates, ULV formulations, and to a lesser extent granular formulations. Sometimes mixtures of solvents are used. The first main groups of solvents are aliphatic paraffinic oils such as kerosene or refined paraffins. The second main group and the most common comprises the aromatic solvents such as xylene and higher molecular weight fractions of C₉ and C₁₀ aromatic solvents. Chlorinated hydrocarbons are useful as cosolvents to prevent crystallization of pesticides when the formulation is emulsified into water. Alcohols are sometimes used as cosolvents to increase solvent power.

Thickeners or gelling agents are used mainly in the formulation of suspension concentrates, emulsions and suspoemulsions to modify the rheology or flow properties of the liquid and to prevent separation and settling of the dispersed particles or droplets. Thickening, gelling, and anti-settling agents generally fall into two categories, namely water-insoluble particulates and water-soluble polymers. It is possible to produce suspension concentrate formulations using clays and silicas. Examples of these types of materials, include, but are limited to, montmorillonite, e.g. bentonite; magnesium aluminum silicate; and attapulgite. Water-soluble polysaccharides have been used as thickening-gelling agents for many years. The types of polysaccharides most commonly used are natural extracts of seeds and seaweeds or are synthetic derivatives of cellulose. Examples of these types of materials include, but are not limited to, guar gum; locust bean gum; carrageenan; alginates; methyl cellulose; sodium carboxymethyl cellulose (SCMC); hydroxyethyl cellulose (HEC). Other types of anti-settling agents are based on modified starches, polyacrylates, polyvinyl alcohol and polyethylene oxide. Another good anti-settling agent is xanthan gum.

Microorganisms which cause spoilage of formulated products. Therefore preservation agents are used to eliminate or reduce their effect. Examples of such agents include, but are limited to propionic acid and its sodium salt; sorbic acid and its sodium or potassium salts; benzoic acid and its sodium salt; p-hydroxy benzoic acid sodium salt; methyl p-hydroxy benzoate; and 1,2-benzisothiazalin-3-one (BIT).

The presence of surfactants, which lower interfacial tension, often causes water-based formulations to foam during mixing operations in production and in application through a spray tank. In order to reduce the tendency to foam, anti-foam agents are often added either during the production stage or before filling into bottles. Generally, there are two types of anti-foam agents, namely silicones and non-silicones. Silicones are usually aqueous emulsions of dimethyl polysiloxane while the non-silicone anti-foam agents are water-insoluble oils, such as octanol and nonanol, or silica. In both cases, the function of the anti-foam agent is to displace the surfactant from the air-water interface.

For further information see “Chemistry and Technology of Agrochemical Formulations” edited by D. A. Knowles, copyright 1998 by Kluwer Academic Publishers. Also see “Insecticides in Agriculture and Environment—Retrospects and Prospects” by A. S. Perry, I. Yamamoto, I. Ishaaya, and R. Perry, copyright 1998 by Springer-Verlag.

### Applications

The actual amount of pesticide to be applied to loci of pests is generally not critical and can readily be determined by those skilled in the art. In general, concentrations from about 0.01 grams of pesticide per hectare to about 5000 grams of pesticide per hectare are expected to provide good control.

The locus to which a pesticide is applied can be any locus inhabited by an pest, for example, vegetable crops, fruit and nut trees, grape vines, ornamental plants, domesticated animals, the interior or exterior surfaces of buildings, and the soil around buildings. Controlling pests generally means that pest populations, activity, or both, are reduced in a locus. This can come about when: pest populations are repulsed from a locus; when pests are incapacitated, partially or completely, temporarily or permanently, in or around a locus; or pests are exterminated, in whole or in part, in or around a locus. Of course a combination of these results can occur. Generally, pest populations, activity, or both are desirably reduce more than fifty percent, preferably more than 90 percent, even more preferably 99 percent.

Generally, with baits, the baits are placed in the ground where, for example, termites can come into contact with the bait. Baits can also be applied to a surface of a building, (horizontal, vertical, or slant, surface) where, for example, ants, termites, cockroaches, and flies, can come into contact with the bait.

Because of the unique ability of the eggs of some pests to resist pesticides repeated applications may be desirable to control newly emerged larvae.

Systemic movement of pesticides in plants may be utilized to control pests on one portion of the plant by applying the pesticides to a different portion of the plant, or to a location where the root system of a plant can uptake pesticides. For example, control of foliar-feeding insects can be controlled by drip irrigation or furrow application, or by treating the seed before planting. Seed treatment can be applied to all types of seeds, including those from which plants genetically transformed to express specialized traits will germinate. Representative examples include those expressing proteins toxic to invertebrate pests, such as Bacillus thuringiensis or other insecticidal toxins, those expressing herbicide resistance, such as “Roundup Ready” seed, or those with “stacked” foreign genes expressing insecticidal toxins, herbicide resistance, nutrition-enhancement or any other beneficial traits. Furthermore, such seed treatments with the invention disclosed in this document can further enhance the ability of a plant to better withstand stressful growing conditions. This results in a healthier, more vigorous plant, which can lead to higher yields at harvest time.

It should be readily apparent that the invention can be used with plants genetically transformed to express specialized traits, such as Bacillus thuringiensis or other insecticidal toxins, or those expressing herbicide resistance, or those with “stacked” foreign genes expressing insecticidal toxins, herbicide resistance, nutrition-enhancement or any other beneficial traits. An example of such a use is spraying such plants with the invention disclosed in this document.

The invention disclosed in this document is suitable for controlling endoparasites and ectoparasites in the veterinary medicine sector or in the field of animal keeping. Compounds according to the invention are applied here in a known manner, such as by oral administration in the form of, for example, tablets, capsules, drinks, granules, by dermal application in the form of, for example, dipping, spraying, pouring on, spotting on, and dusting, and by parenteral administration in the form of, for example, an injection.

The invention disclosed in this document can also be employed advantageously in livestock keeping, for example, cattle, sheep, pigs, chickens, and geese. Suitable formulations are administered orally to the animals with the drinking water or feed. The dosages and formulations that are suitable depend on the species.

Before a pesticide can be used or sold commercially, such pesticide undergoes lengthy evaluation processes by various governmental authorities (local, regional, state, national, international). Voluminous data requirements are specified by regulatory authorities and must be addressed through data generation and submission by the product registrant or by another on the product registrant's behalf. These governmental authorities then review such data and if a determination of safety is concluded, provide the potential user or seller with product registration approval. Thereafter, in that locality where the product registration is granted and supported, such user or seller may use or sell such pesticide.

The headings in this document are for convenience only and must not be used to interpret any portion thereof.

What is claimed is:

1. A compound having the following formula: 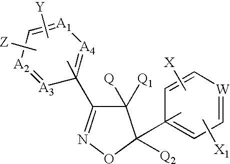
wherein:
A₁, A₂, A₃, and A4 are independently C or N;
Q, Q₁, and Q₂, are independently, H, (C₁-C₆)alkyl, halo(C₁-C₆)alkyl, halo, CN, CO₂R, and CONR₁R₂;
W is C or N;
X and X1 are independently, H, halo, CN, NO₂, (C₁-C₆)alkyl, (C₁-C₆)alkyl-S—, (C₁-C₆)alkoxy, halo(C₁-C₆)alkoxy, halo(C₁-C₆)alkyl-S—, (C₂-C₆)alkenyl, and SCN;
Y and Z are independently, H, halo, CN, NO₂, (C₁-C₆)alkyl, (C₁-C₆)alkyl-S—, (C₁-C₆)alkoxy, halo(C₁-C₆)alkoxy, halo(C₁-C₆)alkyl-S—, (C₂-C₆)alkenyl, SCN, NRR₁, OR₁, or D₁, D₂, D₃, D₄, D₅, D₆, D₇, D₈, D₉, D₁₀, and D₁₁ 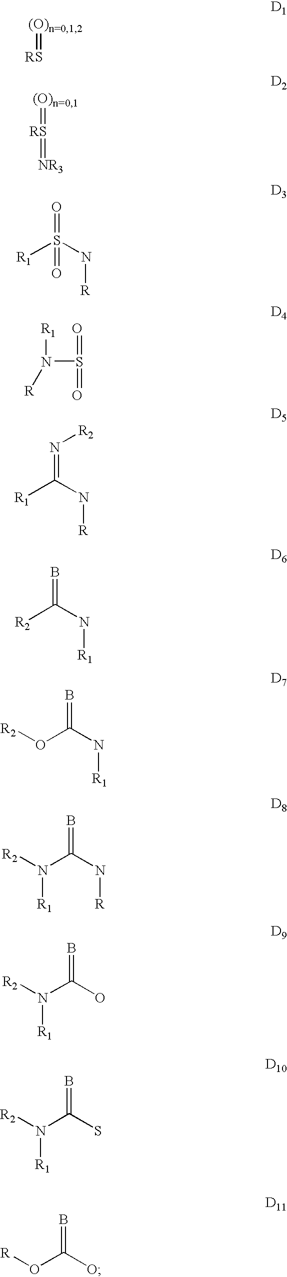
B is O or S;
R, R₁, and R₂, are independently, H, (C₁-C₆)alkyl, halo(C₁-C₆)alkyl, (C₃-C₆)cycloalkyl, (C₂-C₆)alkenyl, halo(C₂-C₆)alkenyl, aryl, heterocyclyl, aryl-alkyl-, heterocyclyl-alkyl-;
R₁ and R₂ can be connected by C, N, O, or S, to form a 3-6 membered ring; and
R₃ can be H, NO₂, CN, COH, COR₁, and CO₂R₁.

2. A compound according to claim 1 wherein Y is D₁ or D₂.

3. A compound according to claim 1 wherein Q and Q₁ are H.

4. A compound according to claim 1 wherein Q₂ is a halo(C₁-C₆)alkyl.

5. A compound according to claim 1 wherein W is C.

6. A compound according to claim 1 wherein X and X₁ are halo.

7. A compound according to claim 1 wherein A₁, A₂, A₃, and A4 are C.

8. A compound according to claim 1 wherein R is a (C₁-C₆)alkyl.

9. A compound according to claim 1 wherein Z and Y are H.

10. A compound according to claim 1 wherein R₃ is H, CN, NO₂, COR₁, or COOR₁.

11. A compound that is a pesticidally acceptable acid addition salt of a compound according to claim 1.

12. A solvate comprising compound according to claim 1.

13. A process comprising applying a compound according to claim 1 to a locus to control pests.

14. A composition comprising a mixture of a compound according to claim 1 with at least one other pesticide.

15. A process of applying a compound of claim 1 to a seed.

16. A process of applying a compound of claim 1 to a seed that has been genetically transformed to express one or more specialized traits.

17. A process of applying a compound of claim 1 to a genetically transformed plant that has been genetically transformed to express one or more specialized traits.

18. A process of orally administering or applying a compound of claim 1 to an animal.

19. A process comprising submitting data relating to a compound of claim 1 to a governmental authority in order to obtain product registration approval for a product comprising a compound of claim 1.
